(19) United States
(12) Patent Application Publication
(10) Pub. No.: US 2005/0276831 A1
(43) Pub. Date: Dec. 15, 2005

(54) Benefit agent containing delivery particle

(76) Inventors: Jiten Odhavji Dihora, Hamilton, OH (US); Martin Ian James, Montesilvano (IT); Johan Smets, Lubbeek (BE)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 11/145,904

(22) Filed: Jun. 6, 2005

Related U.S. Application Data
(60) Provisional application No. 60/578,761, filed on Jun. 10, 2004.
(60) Provisional application No. 60/620,030, filed on Oct. 19, 2004.

(51) Int. Cl. A61K 7/00

(52) U.S. Cl. 424/401

(57) ABSTRACT

The present invention relates to benefit agent containing delivery particles, compositions comprising said particles, and processes for making and using the aforementioned particles and compositions. When employed in compositions, for example cleaning or fabric care compositions, such particles increase the efficiency of benefit agent delivery, there by allowing reduced amounts of benefit agents to be employed. In addition to allowing the amount of benefit agent to be reduced, such particles allow a broad range of benefit agents to be employed.

### CROSS-REFERENCES TO RELATED APPLICATIONS

This application claims priority under 35 U.S.C. § 119(e) to U.S. Provisional Application Ser. No. 60/578,761 filed Jun. 10, 2004 and U.S. Provisional Application Ser. No. 60/620,030 filed Oct. 19, 2004.

### FIELD OF INVENTION

This relates to benefit agent containing delivery particles, compositions comprising such particles, and processes for making and using such particles and compositions.

### BACKGROUND OF THE INVENTION

Benefit agents, such as perfumes, silicones, waxes, flavors, vitamins and fabric softening agents, are expensive and generally less effective when employed at high levels in personal care compositions, cleaning compositions, and fabric care compositions. As a result, there is a desire to maximize the effectiveness of such benefit agents. One method of achieving such object is to improve the delivery efficiencies of such benefit agents. Unfortunately, it is difficult to improve the delivery efficiencies of benefit agents as such agents may be lost do to the agents' physical or chemical characteristics, or such agents may be incompatible with other compositional components or the situs that is treated.

Accordingly, there is a need for a benefit agent containing delivery particle that provides improved benefit agent delivery efficiency.

### SUMMARY OF THE INVENTION

The present invention relates to benefit agent containing delivery particles comprising:
a.) a core material comprising a benefit agent selected from the group consisting of perfume, silicone oils, waxes, hydrocarbons, higher fatty acids, essential oils, lipids, skin coolants, vitamins, sunscreens, glycerine, catalysts, bleach particles, silicon dioxide particles, malodor reducing agents, antiperspirant actives, cationic polymers and mixtures thereof; and
b.) one or more coatings that coat at least a portion of said core material, said one or more coatings having a total work of adhesion of at least 0.2 millijoules, a total percent of dissolution of less than 80%, said benefit agent containing delivery particle having a benefit agent loss of less than 70%.
   The present invention also relates to compositions comprising said particles, and processes for making and using such particles and compositions.

### DETAILED DESCRIPTION OF THE INVENTION

Definitions

As used herein, the term “cleaning composition” includes, unless otherwise indicated, granular or powder-form all-purpose or “heavy-duty” washing agents, especially cleaning detergents; liquid, gel or paste-form all-purpose washing agents, especially the so-called heavy-duty liquid types; liquid fine-fabric detergents; hand dishwashing agents or light duty dishwashing agents, especially those of the high-foaming type; machine dishwashing agents, including the various tablet, granular, liquid and rinse-aid types for household and institutional use; liquid cleaning and disinfecting agents, including antibacterial hand-wash types, cleaning bars, mouthwashes, denture cleaners, dentifrice, car or carpet shampoos, bathroom cleaners; hair shampoos and hair-rinses; shower gels and foam baths and metal cleaners; as well as cleaning auxiliaries such as bleach additives and “stain-stick” or pre-treat types.

As used herein, the term “fabric care composition” includes, unless otherwise indicated, fabric softening compositions, fabric enhancing compositions, fabric freshening compositions and combinations there of.

As used herein, the articles “a” and “an” when used in a claim, are understood to mean one or more of what is claimed or described.

The test methods disclosed in the Test Methods Section of the present application must be used to determine the respective values of the parameters of Applicants' inventions.

Unless otherwise noted, all component or composition levels are in reference to the active portion of that component or composition, and are exclusive of impurities, for example, residual solvents or by-products, which may be present in commercially available sources of such components or compositions.

All percentages and ratios are calculated by weight unless otherwise indicated. All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

All documents cited are, in relevant part, incorporated herein by reference; the citation of any document is not to be construed as an admission that it is prior art with respect to the present invention.

Benefit Agent Containing Delivery Particle

Applicants discovered that the problem of achieving effective and efficient benefit agent delivery can be solved in an economically acceptable manner when the desired benefit agent is coated with one or more coatings having a combination of compositional, physical and chemical characteristics. Thus, while not being bound by theory, Applicants' believe that the recited work of adhesion and percent of dissolution provide deposition efficiency, while proper compositional selection provides barrier characteristics that result in low benefit agent loss levels, which translates to more benefit agent being available when benefit agent release is required.

Applicants' benefit agent containing delivery particle may comprise a core material comprising a benefit agent and a coating that coats at least a portion of said core material. Said coating typically having a work of adhesion of at least 0.2 millijoules, a work of adhesion of from about 0.3 millijoules to about 20 millijoules, or alternatively a work of adhesion from about 0.5 millijoules to about 10 millijoules, and a percent of dissolution of less than about 80%, less than about 50% or alternatively less than about 20%. Said benefit agent containing delivery particle typically has a benefit agent loss of less than about 70%, less than about 50% or alternatively less than about 20%. As a result, such benefit agent containing delivery particles may have benefit agent delivery efficiencies of up to about 80%.

In one aspect of Applicants' invention Applicants' benefit agent containing delivery particle may comprise multiple coatings that are equivalent to said single coating. Said multiple coatings having a total work of adhesion of at least 0.2 millijoules, a total work of adhesion of from about 0.3 millijoules to about 20 millijoules, or alternatively a total work of adhesion from about 0.5 millijoules to about 10 millijoules, and a total percent of dissolution of less than about 80%, less than about 50% or alternatively less than about 20%.

In one aspect of Applicants' invention, said benefit agent containing delivery particle's coating can have a storage modulus from about 5 Pascals to about 500 Pascals, from about 20 Pascals to about 500 Pascals, or alternatively from about 25 Pascals to about 125 Pascals.

In one aspect of Applicants' invention, said benefit agent containing delivery particle should have interfacial tension such that the interfacial tension ratio may be greater than 1 or even greater than 5. While not being bound by theory, it is believed that the aforementioned interfacial tension ratio causes the substrate to prefer the benefit agent containing delivery particle over any delivery medium comprising the benefit agent containing delivery particle.

In one aspect of Applicants' invention, said benefit agent containing delivery particle can have a deposition efficiency of at least 20%, at least 50% or alternatively at least 70%.

In one aspect of Applicants' invention, said benefit agent containing delivery particle can comprise at least 1 weight % benefit agent, from about 5 weight % to about 85 weight %, or alternatively from about 20 weight % to about 70 weight % of a benefit agent.

In one aspect of Applicants' invention, said benefit agent containing delivery particle has a particle size of from about 12 microns to about 2,000 microns, from about 30 microns to about 100 microns or alternatively from about 45 microns to about 75 microns.

In one aspect of Applicants' invention, said particle may comprise a material selected from dyes, pigments and mixtures thereof.

In one aspect of Applicants' invention said core material may comprise a shell and/or adsorbent, at least a portion of said benefit agent being encapsulated by said shell when said shell is present and at least a portion of said benefit agent absorbed into said adsorbent when said adsorbent is present.

In one aspect of Applicants' invention said benefit agent containing delivery particle may have and/or comprise any combination of the parameters of previously described aspects of Applicants' invention.

Useful core shell materials include materials selected from the group consisting of reaction products of one or more amines with one or more aldehydes, such as urea cross-linked with formaldehyde or gluteraldehyde, melamine cross-linked with formaldehyde; gelatin-polyphosphate coacervates optionally cross-linked with gluteraldehyde; gelatin-gum Arabic coacervates; cross-linked silicone fluids; polyamine reacted with polyisocyanates and mixtures thereof.

Useful core material absorbents include materials selected from the group consisting of aluminosilicate particles having a high absorption and/or adsorption efficiency, porous silica materials with high absorption and/or adsorption efficiency, and mixtures thereof. Useful adsorbent materials typically have a total surface area of about 50 square meters per gram to about 650 square meters per gram.

Useful benefit agents include materials selected from the group consisting of perfumes such as 3-(4-t-butylphenyl)-2-methyl propanal, 3-(4-t-butylphenyl)-propanal, 3-(4-isopropylphenyl)-2-methylpropanal, 3-(3,4-methylenedioxyphenyl)-2-methylpropanal, and 2,6-dimethyl-5-heptenal, α-damascone, β-damascone, δ-damascone, β-damascenone, 6,7-dihydro-1,1,2,3,3-pentamethyl-4 (5H)-indanone, methyl-7,3-dihydro-2H-1,5-benzodioxepine-3-one, 2-[2-(4-methyl-3-cyclohexenyl-1-yl)propyl]cyclopentan-2-one, 2-sec-butylcyclohexanone, and β-dihydro ionone, linalool, ethyllinalool, tetrahydrolinalool, and dihydromyrcenol; silicone oils, waxes such as polyethylene waxes; hydrocarbons such as petrolatum; essential oils such as fish oils, jasmine, camphor, lavender; skin coolants such as menthol, methyl lactate; vitamins such as Vitamin A and E; sunscreens; glycerine; catalysts such as manganese catalysts or bleach catalysts; bleach particles such as perborates; silicon dioxide particles; antiperspirant actives; cationic polymers, such as ditallowoyl ethanol ester dimethyl ammonium chloride, and mixtures thereof. Suitable benefit agents can be obtained from Givaudan Corp. of Mount Olive, N.J., USA, International Flavors & Fragrances Corp. of South Brunswick, N.J., USA, or Quest Corp. of Naarden, Netherlands.

Non-limiting examples of coating materials that have the specified work of adhesion and dissolution percent, and which are useful in producing embodiments of Applicants' benefit agent containing delivery particle include 20 mol % to 70 mol % hydrolyzed polyvinyl alcohol; a gel comprising cross-linked, fully hydrolyzed polyvinyl alcohol (4.88 wt % polyvinyl alcohol having an average weight average molecular weight of 40,000 g/mol and 99 mol % hydrolysis, 0.21 wt % hydrochloric acid, 115-125 parts per million by weight gluteraldhehyde, and balance water; 4.88 wt % polyvinyl alcohol having an average weight average molecular weight of 155,000 g/mol and 99 mol % hydrolysis, 0.21 wt % sodium hydroxide, 95-110 parts per million by weight of sodium tetraborate, and balance water); a 20 wt % aqueous solution of high molecular weight polyvinyl pyrrolidone (weight average molecular weight of 1200-2000 kdaltons); or a mixture of 1200-2000 kdalton weight average molecular weight polyvinyl pyrrolidone and polyethylene glycol 400 (average molecular weight of 400 daltons) in the ratio of 15 parts polyvinyl pyrrolidone to 85 parts polyethylene glycol 400 by weight; alternatively, a mixture of 1200-2000 kdalton polyvinyl pyrrolidone and polyethylene glycol 400 in the ratio of 25 parts 1200-2000 kdalton polyvinyl pyrrolidone to 75 parts polyethylene glycol 400 by weight; alternatively, a 40 wt % aqueous solution comprising polyvinyl pyrrolidone, having a molecular weight in the range of 1200-2000 kilodaltons, and ethoxylated alcohols in a ratio of 1:1 on a solids basis. Alternatively, cross-linked polysaccharide materials, e.g. jet cooked Ethylex™ 2065 (a hydroxyethylated dent corn starch) to which is added sodium borate under alkaline conditions to form a polysaccharide gel with a typical composition of 12.7 wt % Ethylex™ 2065, 0.19 wt % sodium borate, 0.46 wt % sodium hydroxide, and balance water. Alternatively, mixtures of these systems can be utilized to achieve the specified work of adhesion, dissolution percent, and benefit agent loss. Such materials can be obtained from Celanese Chemicals Corp. of Dallas, Tex., USA, International Specialty Products Corp. of Wayne, New Jersey, USA, National Starch and Chemical Corp. of Bridgewater, N.J., USA, Dow Chemical Corp. of Midland, Mich., USA, BASF AG of Ludwigshafen, Germany, AE Staley Corp. of Decatur, Ill., USA, Tate & Lyle PLC of London, United Kingdom; Nippon Gohsei of Osaka, Japan; Shell Chemicals Corporation of Westhollow, Tex., USA.

Non-limiting examples of coating materials that have the specified work of adhesion and dissolution percent, and which, in combination with a barrier material can result in embodiments of Applicants' benefit agent containing delivery particle, include materials selected from the group consisting of polyvinyl alcohol, polyvinyl pyrrolidone, polyethylene glycol, polyamines including but not limited to polyaminoalcohols, polysaccharides and modified polysaccharides, gel forming proteins, modified celluloses such as carboxymethylcelluloses and hydroxyethylcelluloses, carboxylic acid containing acrylic polymers, polyureas, polyurethanes and mixtures thereof. Such materials can be obtained from Celanese Chemicals Corp. of Dallas, Tex., USA, International Specialty Products Corp. of Wayne, N.J., USA, Hercules Corp. of Wilmington, Del., USA, National Starch and Chemical Corp. of Bridgewater, N.J., USA, and Dow Chemical Corp. of Midland, Mich., USA, and BASF of Ludwigshafen, Germany.

Non-limiting examples of coating materials that can serve as barrier materials and that in combination with a material having the specified work of adhesion and dissolution percent can result in embodiments of Applicants' benefit agent containing delivery particle include materials selected from the group consisting of polyvinyl pyrrolidone and its various copolymers with styrene, vinyl acetate, imidazole, primary and secondary amine containing monomers, methyl acrylate, polyvinyl acetal, maleic anhydride; polyvinyl alcohol and its various copolymer with 2-acrylamide-2-methylpropane sulfonate, primary and secondary amine containing monomers, imidazoles, methyl acrylate; polyacrylamides; polyacrylic acids; microcrystalline waxes; paraffin waxes; modified polysaccharides such as waxy maize or dent corn starch, octenyl succinated starches, derivatized starches such as hydroxyethylated or hydroxypropylated starches, carrageenan, guar gum, pectin, xanthan gum; modified celluloses such as hydrolyzed cellulose acetate, hydroxy propyl cellulose, methyl cellulose, and the like; modified proteins such as gelatin; hydrogenated and non-hydrogenated polyalkenes; fatty acids; hardened shells such as urea crosslinked with formaldehyde, gelatin-polyphosphate, melamine-formaldehyde, polyvinyl alcohol crosslinked with sodium tetraborate or gluteraldehyde; latexes of styrene-butadiene, ethyl cellulose, and the like; and mixtures thereof. Such materials can be obtained from CP Kelco Corp. of San Diego, Calif., USA; Degussa AG or Dusseldorf, Germany; BASF AG of Ludwigshafen, Germany; Rhodia Corp. of Cranbury, N.J., USA; Baker Hughes Corp. of Houston, Tex., USA; Hercules Corp. of Wilmington, Del., USA; Agrium Inc. of Calgary, Alberta, Canada and International Specialty Products of Wayne, N.J. USA.

Non-limiting examples of dyes and pigments include organic and inorganic pigments, aqueous and other solvent-soluble dyes. Such dyes and pigments can be obtained from Ciba Specialty Chemicals Corp. of Newport, Del., U.S.A.; Clariant Corp. of Charlotte, N.C., U.S.A.; and Milliken Chemical Co. of Spartanburg, S.C., U.S.A.

Process of Making Benefit Agent Containing Delivery Particles

The benefit agent delivery particle disclosed in the present application may be made via the teachings and examples disclosed herein.

Typically, the benefit agent delivery particle disclosed in the present application may be made by applying one or more coatings having, a total work of adhesion of at least 0.2 millijoules, a total work of adhesion of from about 0.3 millijoules to about 20 millijoules, or alternatively a total work of adhesion from about 0.5 millijoules to about 10 millijoules, and a total percent of dissolution of less than about 80%, less than about 50% or alternatively less than about 20% to a core material that may comprise a benefit agent to form a benefit agent containing delivery particle, said one or more coatings being applied in a sufficient amount to provide said benefit agent containing delivery particle with a benefit agent loss of less than about 70%, less than about 50% or alternatively less than about 20%.

In one aspect of Applicants' invention, said one or more coatings may have an interfacial tension such that the interfacial tension ratio may be greater than 1 or even greater than 5.

In one aspect of Applicants' invention any cross-linking of the coating materials is performed prior to said coating step.

In one aspect of Applicants' invention, benefit agent delivery particles are made by a coacervation process that may optionally be followed by a spray drying process. Such process may comprise the steps of dispersing the benefit agent or benefit agent contained within a barrier particle in a slurry of the adhesive coating material. A suspending agent (e.g. carboxymethyl cellulose) is added, followed by the addition of an anti-solvent (e.g. sodium sulfate) to cause a phase separation, and precipitation of the adhesive coating material onto the dispersed phase. The resulting slurry may be used as is or optionally atomized in a spray dryer to obtain dry particles.

In another aspect of Applicants' invention, benefit agent containing delivery particles are made by an extrusion process. Such process may comprise the steps of mixing the benefit agent with the adhesive coating material in a twin screw extruder or a co-extruder to form a slurry that is passed through a die to yield noodles. The noodles can be ground to a powder of the desired particle size. A starch melt, that can improve the controlled release properties, can be added during the mixing/extrusion step.

In another aspect of Applicants' invention, benefit agent containing delivery particles are made by an agglomeration process or a fluid bed coating process. Such agglomeration or fluid bed process may comprise the steps of spraying a slurry of adhesive coated benefit agent particles (made by a coacervation process as described above) on to a fluidized bed of carrier particles. The carrier particles may have a diameter 5 times or alternatively 9 times that of the diameter of the particles in the spray slurry.

In another aspect of Applicants' invention, the coating material is added directly during the manufacture of the benefit agent containing delivery particles. Such a process may comprise the steps of forming an emulsion of the benefit agent, then depositing a wall on the surface of the benefit agent, and adjusting the reaction conditions to crosslink the wall material. The coating material may be added to the slurry containing benefit agent during the cross-linking reaction step. Alternatively, the coating material may be added at the last stage of the manufacture of the benefit agent containing particle, together with an agent that can covalently link the coating material to the surface of the benefit agent containing delivery particle. While not being bound by theory, it is believed that such a mode of addition minimizes the dissolution of the coating material and also substantially reduces the quantity of coating material required to achieve the delivery efficiency of particles.

In one aspect of Applicants' invention said benefit agent containing delivery particle may be produced using any combination of the process steps previously described.

Suitable equipment for use in the processes disclosed herein may include paddle mixers, ploughshear mixers, ribbon blenders, vertical axis granulators and drum mixers, both in batch and, where available, in continuous process configurations, spray dryers, and extruders. Such equipment can be obtained from Lodige GmbH (Paderborn, Germany), Littleford Day, Inc. (Florence, Ky., U.S.A.), Forberg AS (Larvik, Norway), Glatt Ingenieurtechnik GmbH (Weimar, Germany), Niro (Soeborg, Denmark), Hosokawa Bepex Corp. (Minneapolis, Minn., USA).

Compositions Comprising Benefit Agent Containing Delivery Particles

The compositions of the present invention may comprise an embodiment of the benefit agent containing delivery particle disclosed in the present application. While the precise level of benefit agent containing delivery particle that is employed depends on the type and end use of the composition, in one aspect of Applicants' invention, a cleaning composition may comprise, based on total cleaning composition weight, from about 0.01 weight % to about 10 weight %, from about 0.1 weight % to about 3 weight %, or alternatively from about 0.5 weight % to about 1.5 weight % of a benefit agent containing delivery particle. In one aspect of Applicants' invention, a fabric treatment composition may comprise from about 0.01 weight % to about 10 weight %, from about 0.1 weight % to about 3 weight %, or alternatively from about 0.5 weight % to about 1.5 weight % of a benefit agent containing delivery particle.

The cleaning compositions disclosed herein are typically formulated such that, during use in aqueous cleaning operations, the wash water will have a pH of between about 6.5 and about 12, or between about 7.5 and 10.5. Liquid dishwashing product formulations typically have a pH between about 6.8 and about 9.0. Cleaning products are typically formulated to have a pH of from about 7 to about 12. Techniques for controlling pH at recommended usage levels include the use of buffers, alkalis, acids, etc., and are well known to those skilled in the art.

Adjunct Materials

While not essential for the purposes of the present invention, the non-limiting list of adjuncts illustrated hereinafter are suitable for use in the instant compositions and may be desirably incorporated in certain embodiments of the invention, for example to assist or enhance performance, for treatment of the substrate to be cleaned, or to modify the aesthetics of the cleaning composition as is the case with perfumes, colorants, dyes or the like. It is understood that such adjuncts are in addition to the components that are supplied via Applicants' delivery particles. The precise nature of these additional components, and levels of incorporation thereof, will depend on the physical form of the composition and the nature of the cleaning operation for which it is to be used. Suitable adjunct materials include, but are not limited to, surfactants, builders, chelating agents, dye transfer inhibiting agents, dispersants, enzymes, and enzyme stabilizers, catalytic materials, bleach activators, polymeric dispersing agents, clay soil removal/anti-redeposition agents, brighteners, suds suppressors, dyes, perfumes, structure elasticizing agents, fabric softeners, carriers, hydrotropes, processing aids and/or pigments. In addition to the disclosure below, suitable examples of such other adjuncts and levels of use are found in U.S. Pat. Nos. 5,576,282, 6,306,812 B1 and 6,326,348 B1 that are incorporated by reference.

As stated, the adjunct ingredients are not essential to Applicants' cleaning and fabric care compositions. Thus, certain embodiments of Applicants' cleaning and fabric care compositions do not contain one or more of the following adjuncts materials: bleach activators, surfactants, builders, chelating agents, dye transfer inhibiting agents, dispersants, enzymes, and enzyme stabilizers, catalytic metal complexes, polymeric dispersing agents, clay and soil removal/anti-redeposition agents, brighteners, suds suppressors, dyes, perfumes, structure elasticizing agents, fabric softeners, carriers, hydrotropes, processing aids and/or pigments. However, when one or more adjuncts is present, such one or more adjuncts may be present as detailed below:

Surfactants—The cleaning compositions and fabric care compositions according to the present invention can comprise a surfactant or surfactant system wherein the surfactant can be selected from nonionic and/or anionic and/or cationic surfactants and/or ampholytic and/or zwitterionic and/or semi-polar nonionic surfactants.

The surfactant is typically present at a level of from about 0.1%, preferably about 1%, more preferably about 5% by weight of the cleaning compositions to about 99.9%, preferably about 80%, more preferably about 35%, most preferably about 30% by weight of the cleaning compositions.

Builders—The cleaning compositions and fabric care compositions of the present invention can comprise one or more detergent builders or builder systems. When present, the compositions will typically comprise at least about 1% builder, preferably from about 5%, more preferably from about 10% to about 80%, preferably to about 50%, more preferably to about 30% by weight, of detergent builder.

Builders include, but are not limited to, the alkali metal, ammonium and alkanolammonium salts of polyphosphates, alkali metal silicates, alkaline earth and alkali metal carbonates, aluminosilicate builders polycarboxylate compounds. ether hydroxypolycarboxylates, copolymers of maleic anhydride with ethylene or vinyl methyl ether, 1,3,5-trihydroxy benzene-2,4,6-trisulphonic acid, and carboxymethyloxysuccinic acid, the various alkali metal, ammonium and substituted ammonium salts of polyacetic acids such as ethylenediamine tetraacetic acid and nitrilotriacetic acid, as well as polycarboxylates such as mellitic acid, succinic acid, oxydisuccinic acid, polymaleic acid, benzene 1,3,5-tricarboxylic acid, carboxymethyloxysuccinic acid, and soluble salts thereof.

Chelating Agents—The cleaning compositions and fabric care compositions herein may also optionally contain one or more copper, iron and/or manganese chelating agents.

If utilized, these chelating agents will generally comprise from about 0.1% by weight of the cleaning compositions herein to about 15%, more preferably from about 3.0% to about 15% by weight of the cleaning compositions herein.

Dye Transfer Inhibiting Agents—The cleaning compositions and fabric care compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof.

When present in the cleaning compositions herein, the dye transfer inhibiting agents are present at levels from about 0.0001%, more preferably about 0.01%, most preferably about 0.05% by weight of the cleaning compositions to about 10%, more preferably about 2%, most preferably about 1% by weight of the cleaning compositions.

Dispersants—The cleaning compositions and fabric care compositions of the present invention can also contain dispersants. Suitable water-soluble organic materials are the homo- or co-polymeric acids or their salts, in which the polycarboxylic acid may comprise at least two carboxyl radicals separated from each other by not more than two carbon atoms.

Enzymes—The cleaning compositions and fabric care compositions can comprise one or more detergent enzymes which provide cleaning performance and/or fabric care benefits. Examples of suitable enzymes include, but are not limited to, hemicellulases, peroxidases, proteases, cellulases, xylanases, lipases, phospholipases, esterases, cutinases, pectinases, keratanases, reductases, oxidases, phenoloxidases, lipoxygenases, ligninases, pullulanases, tannases, pentosanases, malanases, β-glucanases, arabinosidases, hyaluronidase, chondroitinase, laccase, and amylases, or mixtures thereof. A typical combination is cocktail of conventional applicable enzymes like protease, lipase, cutinase and/or cellulase in conjunction with amylase.

Enzyme Stabilizers—Enzymes for use in detergents can be stabilized by various techniques. The enzymes employed herein can be stabilized by the presence of water-soluble sources of calcium and/or magnesium ions in the finished compositions that provide such ions to the enzymes.

Catalytic Metal Complexes—Applicants' cleaning compositions and fabric care compositions may include catalytic metal complexes. One type of metal-containing bleach catalyst is a catalyst system comprising a transition metal cation of defined bleach catalytic activity, such as copper, iron, titanium, ruthenium, tungsten, molybdenum, or manganese cations, an auxiliary metal cation having little or no bleach catalytic activity, such as zinc or aluminum cations, and a sequestrate having defined stability constants for the catalytic and auxiliary metal cations, particularly ethylenediaminetetraacetic acid, ethylenediaminetetra (methylenephosphonic acid) and water-soluble salts thereof. Such catalysts are disclosed in U.S. Pat. No. 4,430,243 Bragg, issued Feb. 2, 1982.

If desired, the compositions herein can be catalyzed by means of a manganese compound. Such compounds and levels of use are well known in the art and include, for example, the manganese-based catalysts disclosed in U.S. Pat. No. 5,576,282 Miracle et al.

Cobalt bleach catalysts useful herein are known, and are described, for example, in U.S. Pat. No. 5,597,936 Perkins et al., issued Jan. 28, 1997; U.S. Pat. No. 5,595,967 Miracle et al., Jan. 21, 1997. Such cobalt catalysts are readily prepared by known procedures, such as taught for example in U.S. Pat. No. 5,597,936, and U.S. Pat. No. 5,595,967.

Compositions herein may also suitably include a transition metal complex of a macropolycyclic rigid ligand—abreviated as “MRL”. As a practical matter, and not by way of limitation, the compositions and cleaning processes herein can be adjusted to provide on the order of at least one part per hundred million of the benefit agent MRL species in the aqueous washing medium, and will preferably provide from about 0.005 ppm to about 25 ppm, more preferably from about 0.05 ppm to about 10 ppm, and most preferably from about 0.1 ppm to about 5 ppm, of the MRL in the wash liquor.

Preferred transition-metals in the instant transition-metal bleach catalyst include manganese, iron and chromium. Preferred MRL's herein are a special type of ultra-rigid ligand that is cross-bridged such as 5,12-diethyl-1,5,8,12-tetraazabicyclo[6.6.2]hexadecane.

Suitable transition metal MRLs are readily prepared by known procedures, such as taught for example in WO 00/332601, and U.S. Pat. No. 6,225,464.

Processes of Making and Using Cleaning Compositions

The cleaning compositions and fabric care of the present invention can be formulated into any suitable form and prepared by any process chosen by the formulator, non-limiting examples of which are described in U.S. Pat. No. 5,879,584 Bianchetti et al., issued Mar. 9, 1999; U.S. Pat. No. 5,691,297 Nassano et al., issued Nov. 11, 1997; U.S. Pat. No. 5,574,005 Welch et al., issued Nov. 12, 1996; U.S. Pat. No. 5,569,645 Dinniwell et al., issued Oct. 29, 1996; U.S. Pat. No. 5,565,422 Del Greco et al., issued Oct. 15, 1996; U.S. Pat. No. 5,516,448 Capeci et al., issued May 14, 1996; U.S. Pat. No. 5,489,392 Capeci et al., issued Feb. 6, 1996; U.S. Pat. No. 5,486,303 Capeci et al., issued Jan. 23, 1996 all of which are incorporated herein by reference.

Method of Use

Compositions containing the benefit agent delivery particle disclosed herein can be used to clean or treat a situs inter alia a surface or fabric. Typically at least a portion of the situs is contacted with an embodiment of Applicants' composition, in neat form or diluted in a liquor, for example, a wash liquor and then the situs may be optionally washed and/or rinsed. For purposes of the present invention, washing includes but is not limited to, scrubbing, and mechanical agitation. The fabric may comprise most any fabric capable of being laundered or treated in normal consumer use conditions. Liquors that may comprise the disclosed cleaning or fabric care compositions may have a pH of from about 3 to about 11.5. Such compositions are typically employed at concentrations of from about 500 ppm to about 15,000 ppm in solution. When the wash solvent is water, the water temperature typically ranges from about 5° C. to about 90° C. and, when the situs comprises a fabric, the water to fabric ratio is typically from about 1:1 to about 30:1.

### Test Methods

It is understood that the test methods that are disclosed in the Test Methods Section of the present application must be used to determine the respective values of the parameters of Applicants' inventions as such inventions are described and claimed herein.
1.) Work of Adhesion Test: measurement of the energy required to break the adhesive bond between a substrate and the adhesive coating material.
   a.) Obtain 10 g of the material that will be tested. Such 10 g of material should be neat. For example, if such material comprises a solvent, the unbound solvent must be separated from the material prior to testing.
   b.) Measure the work of adhesion using a TA X2 Plus Texture Analyzer, which can be obtained from Texture Technologies, as follows: Place the 10 g sample of material in a 4 oz cylindrical glass jar having a diameter of 2 inches. Next, place the jar on the texture analyzer apparatus test stand. Test conditions are as follows:
      (i) Probe: stainless steel 5 mm diameter spherical, covered with a sock of wetted polycotton (50% cotton & 50% polyester cloth wetted for 1 minute in deionized water).
      (ii) Contact time of substrate with adhesive: 90 seconds
      (iii) Penetration rate of fabric coated probe into adhesive sample: 0.5 mm/sec
      (iv) Withdrawal Rate: 10 mm/sec
      (v) Applied force: high enough applied force so that the probe is at least 5 mm into the sample to be tested, typically 0.02N
      (vi) Temperature: 22-24° C.
   c.) Plot force vs. distance traveled by probe—the work of adhesion is reported in millijoules and is the area under the force vs. distance curve.
2.) Storage Modulus Test
   a.) Obtain 10 g of the material that will be tested. Such 10 g of material should be neat. For example, if such material comprises a solvent, the unbound solvent must be separated from the material before to prior testing.
   b.) Measure the storage modulus (G′) by employing oscillatory rheometry using a Carri Med CSL 2-100 rheometer (TA Instruments of New Castle, Del., USA), coupled with a Isotemp Refrigerant Circulator (Fisher Scientific, Pittsburgh, Pa., USA) with the following setup parameters
      (i) Probe: 4 centimeter, 2 degree steel cone geometry
      (ii) 52 micrometer gap between sample and probe
      (iii) Frequency=1.0 Hz, Shear Stress=10 Pascals
      (iv) Temperature of 20° C.
      (v) Test Time=30 seconds
   c.) Record the storage modulus reported by the instrument.
3.) Percent of Dissolution Test, Benefit Agent Loss Test
a.) Add 5.0 g of benefit agent containing delivery particle to 500 g deionized water.
b.) Agitate the slurry at 80 RPM using a 3 blade turbine agitator (1″ pitched blade) obtained from Cole Parmer Inc., for 30 minutes at 20° C.
c.) Filter the slurry through a 0.45 micron Teflon syringe filter (25 mm diameter, 3.9 cm² effective filtration area, 4.1 bar operating pressure, polypropylene housing).
(i) Determine the adhesive coating material concentration (wt %) in the liquid retentate to an accuracy of ±10 percent.
(ii) Calculate the % dissolution by

$$100 * \frac{500 g * ( \begin{matrix}adhesive coating material \\ concentration in wt \%\end{matrix} ) }{5 g * ( wt \% of adhesive coating on benefit agent particle )}$$
4.) Benefit Agent Loss Test.
   a.) Add 5.0 g of benefit agent containing delivery particle to 500 g deionized water.
   b.) Agitate the slurry at 80 RPM using a 3 blade turbine agitator (1″ pitched blade) for 30 minutes at 20° C.
   c.) Filter the slurry through a 0.45 micron Teflon syringe filter (25 mm diameter, 3.9 cm2 effective filtration area, 4.1 bar operating pressure, polypropylene housing).

(i) Determine the benefit agent concentration in the liquid retentate (wt %) to an accuracy of +10 percent. (for example, when perfume is the benefit agent, Gas Chromatography/Mass Spectrometry may be used to determine the concentration. GC Conditions (Hewlett Packard 5890 model II, with flame ionisation detector and equipped with a split injector for capillary G.C. and Millipore Millenium data system, G.C. column. Fused silica, 30 m long×0.32 mm inner diameter, coated with DB-5 stationary phase 0.25 micrometers thick).

| | |
|---|---|
| Carrier gas | Helium |
| Column head pressure | 9 psi |
| Injection temperature | 300° C. |
| Injection split ratio | 20:1 |
| Detector temperature | 320° C. |
| Oven programme | 50° C. to 300° C. at 7° C./min |

d.) Calculate the % benefit agent loss by

$$100 * \frac{500 g * ( \begin{matrix}benefit agent concentration \\ in liquid retentate in wt \%\end{matrix} ) }{5 g * ( \begin{matrix}wt \% of benefit agent in benefit \\ agent containing particle\end{matrix} )}$$
5.) Deposition Efficiency Test: this test comprises three steps. The first step is to prepare benefit agent coated particles with a tracer (e.g. an element that is not present on the fabrics or the cleaning composition to be utilized). The second step is to perform a wash test by adding a known amount of benefit agent containing particle to a washing process, followed by sufficient drying of the substrate. The third step is to collect representative samples of the washed substrate, and measure the elemental composition. The washing method may be different based on the benefit to be delivered; however an example laundering method is provided below illustrating these steps.
   a.) Prepare the benefit agent containing delivery particle with a tracer: incorporate zeolite 13× supplied by UOP LLC in the particle preparation process at a level of 10 wt % of the particle (15 wt % Aluminum, 27 wt % Silicon in this zeolite). Alternatively, one can incorporate a colloidal silica dispersion (Ludox HS-30 from Grace Davison of Maryland, USA) at a level of 10 wt % of the particle.
   b.) Perform a laundering wash test by adding 2.5 g of the benefit agent containing delivery particle to a washer having a diameter of 10 inches, height of 8.5 inches, and an agitator having a diameter of 1.5 inches, height of 9.5 inches, and base of 6 inches. The washer is filled with 7 liters of deionized water at 32° C., 7 cotton terry fabrics (12″×12″ wash towels composed of 86% polycotton, 14% polyester EuroSoft brand cotton terries manufactured by Silara Exports Ltd of India). Powder detergent having a composition similar to that listed in Example 11 (but nil zeolite) is added to the washing machine. The washing cycle comprises: 12 minutes wash at 80 RPM agitation, 2 minute spin cycle, 2 minute rinse cycle with water at 16° C., followed by a 2 minute spin cycle. The fabrics are then machine dried in a tumble dryer for 45 minutes.
   c.) At least 3 separate wash trials must be conducted with the benefit agent containing delivery particle. Six representative fabric swatches (0.50 grams each) are collected. The fabrics are digested with nitric acid and hydrofluoric acid. To 0.50 grams of fabric is added 10.00 mL of concentrated nitric acid, and 0.200 μL of hydrofluoric acid. The sample is digested using a Ethos Plus Microwave Labstation (manufactured by Milestone, Inc. of Monroe, Conn., USA) using the following 4-cycle program: 3 minutes at 85° C. maximum power of 700 Watts, 9 minutes at 145° C. maximum power of 500 Watts, 4 minutes at 200° C. maximum power 1000 Watts, 14 minutes at 200° C. maximum power 1000 Watts.
   d.) The digested fabrics are then diluted 50 to 1 with deionized water. The diluted slurry is fed to a Perkin Elmer Optima 4300 DV spectrometer (by Perkin Elmer of Chicago, Ill., USA) and analyzed for the desired elements (silicon, aluminum) at the following instrument settings: Plasma 15 Liters per minute, Auxilary 0.5 Liters per minute, Nebulizer at 0.8 Liters per minute, Power of 1400 Watts, View Distance of 15 millimeters, Axial plasma view, wet aerosol analysis with 1.50 milliliters per minute of slurry flow via a peristaltic pump, 20 second flush time, with a quality control check every 10 samples.
   e.) The reported microgram element per gram substrate reported by the instrument is converted to a deposition efficiency by first subtracting the elemental composition of a control fabric (zero quantity of zeolite added), next multiplying the reported number by the total mass of fabric added to the washing machine (260 grams), then divided the quantity of element that is added to the washing machine (0.15 grams of Aluminum, and 0.27 grams of Silicon in 1 gram of zeolite 13×), and finally multiplying by 100 to obtain a deposition efficiency as a percent.
6.) Core Material Absorbent (BET) Surface Area Measurement
   Core material absorbent surface area is determined in accordance with the (BET) Surface Area Measurement protocol found in the Journal of the American Chemical Society, Volume 60, 1938, p 309.
7.) Interfacial Tension and Interfacial Tension Ratio: Separately measure the contact angle of the benefit agent containing delivery particle and the substrate to which said benefit agent containing delivery particle will be applied using a Kruss K100 Tensiometer according the protocol described in the Washburn Method (Washburn, E. W.; Phys. Rev., 17, 374, 1921) with the following exceptions:
   a.) Benefit Agent Containing Delivery Particle: Place a 0.45 micron filter paper at the bottom of a Kruss sample holder FL12. Add known mass of powder to the FL12, and place another filter paper on top of the powder. Next, screw down the piston of the sample holder. There should be enough mass of particle such that there is resistance during the last rotation of the piston screw.
   b.) Substrate: Saturate the substrate with D.I. water and then immediately measure such substrate's contact angle.
      Once the substrate and benefit agent containing delivery particle are prepared, use the tensiometer to assess the contact angle of the benefit agent containing delivery particle and substrate samples to three separate probe fluids: hexane, ethylene glycol, and diiodomethane. From the contact angle data, and known polar & dispersive components of surface tension of the probe liquids, use Fowkes equation (Fowkes, F. M.; Industrial and Engineering Chemistry, vol. 56, No. 12, page 40, 1964) to calculate the polar and dispersive components of the substrate and benefit agent containing delivery particle. Use Good's equation (Good, R. J., Grifalco, L. A.; J. Phys. Chem., vol. 64, page 561, 1960) to calculate the interfacial tension of the D.I. water wetted substrate, and the benefit agent containing delivery particle.
      Interfacial Tension Ratio is defined as:

### Interfacial Tension of Substrate

### Interfacial Tension of Benefit Agent Containing Delivery Particle

### EXAMPLES

### Example 1

### Preparation of Adhesive Coating Material

A 10 wt % solution of polyvinyl alcohol is made by dissolving 100 grams of granular polyvinyl alcohol in 900 grams of deionized water initially at 95° C., using an Ultra Turrax T-25 mixer at 19000 RPM for 10 minutes to yield a clear solution. The solution is allowed to cool to 30° C., and 1.08 g of 50 wt % gluteraldehyde (Mallinkrodt Baker, Phillipsburg, N.J.) crosslinking agent is added to the mix, followed by addition of 23.0 grams of 2.0M HCl to obtain a final solution pH of 3. The solution is left to sit overnight for adhesive gel formation.

### Example 2

### Method of Making Benefit Agent Containing Delivery Particle (Benefit Agent+Adhesive Coating)

To 163 g of the adhesive gel of Example 1 made with Celvol 107 (Celanese Chemicals, Dallas, Tex.) is added 152 g of deionized water. The phase separated dispersion is mixed using an Ultra-Turrax mixer to obtain a homogeneous dispersion. 158 g of a 1 wt % Aqualon carboxymethyl cellulose 7M8SFPH solution (Hercules, Inc. Wilmington, Del.) is added to the slurry. 77 g of perfume is added to the mixture under agitation, and an emulsion is formed with an approximate mean particle size of 10 micrometers. To the emulsion is added 53 g of 20 wt % sodium sulfate solution (Prior Chemical Corporation, New York, N.Y.) dropwise in order to initiate precipitation of the polyvinyl alcohol onto the dispersed oil phase.

### Example 3

### Method of Making Benefit Agent Containing Delivery Particle (Benefit Agent+Adhesive Coating+Barrier Coating)

To 602 grams of the slurried adhesive coated benefit agent particles of Example 2 is added 90 g of a 50 wt % solution of Maltodextrin CR-10 (Archer Daniels Midland, Decatur, Ill.). The mixture is spray dried using a co-current Niro 3 ft diameter spray dryer operating with a 2 inch diameter spinning wheel atomizer, at the following operating conditions: inlet air temperature of 200 degrees Centigrade, outlet temperature of 95° C. to 98° C., 80 kg/hr air flow rate, disk speed of 30,000 RPM, and a dryer operating pressure of −200 mm H₂O. The particles collected from the dryer have a mean particle size of 50 micrometers.

### Example 4

### Method of Making Benefit Agent Containing Delivery Particle (Benefit Agent+Barrier Coating+Adhesive Coating)

To 158 g of the 10 wt % gel of Example 1 made with Celvol 107 (Celanese Chemicals, Dallas, Tex.) is added 160 g of water, and the gel is homogeneously dispersed using a rotor-stator Ultra Turrax T25 mixer. 158 g of a 1 wt % carboxymethyl cellulose 7M8SFPH (Hercules, Inc. Wilmington, Del.) is added to the dispersed adhesive, followed by the addition of 77 g of PMU capsules (Aveka, Inc. Woodbury, Minn.). The capsules are approximately 50 micrometers in diameter, and contain a 80 wt % fragrance oil core, and 20 wt % urea-formaldehyde wall. While agitating, 53 g of a 20 wt % solution of sodium sulfate (Prior Chemical Corporation, New York, N.Y.) is added dropwise to induce phase separation of the adhesive onto the PMU particles. The contents are spray dried, the particles collected are found to have a mean particle size of 70 micrometers.

### Example 5

### Method of Making Benefit Agent Particles

The particles of Example 2, where the only change is the addition of 150 g of Ludox HS-30 Colloidal Silica (Grace Davison, Chattanooga, Tenn.), and the slurry is spray dried using identical conditions to those used in Example 3.

### Example 6

### Method of Making Benefit Agent Particles

The particles of Example 3, where the only change is the addition of 150 g of Ludox HS-30 Colloidal Silica (Grace Davison, Chattanooga, Tenn.) prior to spray drying.

### Example 7

### Method of Making Benefit Agent Particles

The particles of Example 4, where the only change is the addition of 150 g of Ludox HS-30 Colloidal Silica (Grace Davison, Chattanooga, Tenn.) prior to spray drying.

### Example 8

### Benefit Agent Encapsulated in Zeolite Carrier, Coated with Adhesive via Extrusion

16 g of fragrance oil is sprayed onto 64 g of zeolite 13× (UOP LLC, Des Plaines, Ill.), this powder is added to 206 g of the gel of Example 1 made with Sigma Aldrich polyvinyl alcohol (31000-50000 g/mol, 99 mol % hydrolyzed), and contents are extruded to yield noodles. The noodles are dried overnight at ambient temperature. The hardened material is ground into fine powder using a coffee grinder. Particles are sieved to collect <75 micrometer fraction.

### Example 9

### Zeolite Coated with Adhesive Further Coated with Barrier Material

The <75 micrometer particles of Example 8 are added to 267 g of a 25 wt % aqueous solution of FiCAP 100 modified starch (National Starch & Chemical, Bridgewater, N.J.), and spray dried using a co-current Niro dryer under identical conditions as in Example 3.

### Example 10

### An Agglomeration Process to Incorporate Perfume in a Cleaning Composition

4.0 g of the slurry of Example 4 prior to spray drying is added dropwise to a food processor containing a mixture of 10 g magnesium sulfate, 89 g of sodium tripolyphosphate, and 1.0 g of sodium polyacrylate. Such materials can be obtained from Aldrich of Milwaukee, Wis. U.S.A. and BASF AG of Ludwigshafen, Germany.

### Example 11

### Cleaning Compositions Containing Benefit Agent Containing Delivery Particle

The particles of the previous Examples are added to a nil perfume containing dry laundry detergent powders at a level of 0.30 wt % of the detergent powder, 0.15 wt % of the detergent powder, and 0.075 wt % of the detergent powder thus resulting in cleaning compositions having the following formulae.

| | Formulation Examples: | | | | | | | |
|---|---|---|---|---|---|---|---|---|
| | F1 | F2 | F3 | F4 | F5 | F6 | F7 | F8 |
| Delivery particle type (Example #) | None | 4 | 4 | 4 | 5 | 6 | 7 | 8 |
| Delivery particle parts: | 0.150 | 0.300 | 0.150 | 0.040 | 0.76 | 1.27 | 0.75 | 1.43 |
| Formulation balance: | | | | | | | | |
| Sodium alkylbenzenesulfonate | 19.99 | 6.10 | 8.19 | 8.48 | 0.07 | 3.41 | 17.45 | 17.45 |
| Sodium alkylsulfate | 1.16 | 12.20 | 5.13 | 6.08 | 15.27 | 13.71 | 0.00 | 0.00 |
| Ethoxylated sodium alkylsulfate | 0.29 | 0.00 | 0.00 | 0.00 | 0.00 | 0.00 | 1.55 | 1.55 |
| Sodium Percarbonate | 6.16 | 6.16 | 0.00 | 3.49 | 2.78 | 4.50 | 11.67 | 3.21 |
| Nonanoyloxybenzenesulfonate | 4.75 | 4.75 | 2.10 | 2.41 | 1.92 | 5.16 | 0.00 | 0.00 |
| Tetraacetylethylenediamine | 0.00 | 0.00 | 0.00 | 0.00 | 0.00 | 0.00 | 2.10 | 2.10 |
| Sodium aluminosilicate hydrate | 13.84 | 12.96 | 25.38 | 27.98 | 32.46 | 32.46 | 14.36 | 12.80 |
| Acrylic/Maleic Acids | 6.35 | 3.36 | 0.00 | 0.00 | 0.00 | 0.00 | 2.30 | 2.30 |
| Copolymer | | | | | | | | |
| Sodium Polyacrylate | 0.00 | 0.00 | 1.51 | 1.53 | 1.74 | 1.18 | 0.00 | 0.00 |
| Sodium Carbonate | 19.55 | 22.25 | 22.48 | 21.47 | 24.11 | 23.33 | 20.60 | 20.60 |
| Sodium Tripolyphosphate | 0.00 | 0.00 | 0.00 | 0.00 | 0.00 | 0.00 | 0.00 | 12.40 |
| Sodium Silicate | 2.43 | 2.47 | 0.00 | 0.00 | 0.00 | 0.00 | 0.00 | 0.00 |
| Sodium | 0.00 | 0.00 | 0.72 | 0.80 | 0.72 | 0.54 | 0.54 | 0.54 |
| diethylenetriaminepentaacetate | | | | | | | | |
| Brightener 15 | 0.17 | 0.17 | 0.00 | 0.11 | 0.08 | 0.12 | 0.12 | 0.12 |
| Brightener 49 | 0.09 | 0.09 | 0.00 | 0.00 | 0.00 | 0.00 | 0.00 | 0.00 |
| Sodium Xylene Sulfonate | 1.81 | 0.00 | 0.00 | 0.00 | 0.00 | 0.00 | 0.00 | 0.00 |
| Polydimethylsiloxane | 0.06 | 0.06 | 0.02 | 0.02 | 0.02 | 0.04 | 0.04 | 0.04 |
| Ethyl Methyl Cellulose | 0.00 | 0.00 | 1.11 | 0.00 | 1.11 | 0.00 | 0.00 | 0.00 |
| Imideazole Epichlorohydrin | 0.00 | 0.00 | 0.15 | 0.00 | 0.15 | 0.00 | 0.00 | 0.00 |
| Savinase active enzyme | 0.054 | 0.054 | 0.015 | 0.010 | 0.015 | 0.021 | 0.021 | 0.021 |
| Carezyme active enzyme | 0.000 | 0.000 | 0.003 | 0.000 | 0.000 | 0.000 | 0.000 | 0.000 |
| Perfume | 0.15 | 0 | 0 | 0 | 0.38 | 0.24 | 0.24 | 0.24 |
| Balance sodium sulfate | | | | | | | | |
| Total formulation = 100.00 | | | | | | | | |

### Example 12

64 g of a 70 wt % emulsion of polydimethylsiloxane (600,000 Centistoke fluid, Dow Chemical of Midland, Mich., USA) is added to a mixture comprising 60 g of a 1 wt % solution of 7M8SFPH Carboxymethylcellulose (Hercules, Inc. of Wilmington, Del.) and 15 g of Ludox HS-30 colloidal silica (Grace Davison, Chattanooga, Tenn.). 100 g of a 5 wt % gel of polyvinyl alcohol (97.946 g a 5 wt % aqueous PVOH solution+0.054 g of 50 wt % gluteraldehyde+2.0 g of 2.0M HCl) is added to the mix, and agitated vigorously at 13,000 RPM using an Ultra Turrax T-25 mixer (DivTech, Inc). 25 g of a 20 wt % sodium sulfate solution is added to the mixture over a period of 3 minutes, under 1100 RPM agitation using a 3-blade agitator (Laboretechnik IKA mixer). 2.9 g of the polydimethylsiloxane slurry is then added to a miniwash cycle, and cotton terry fabrics are analyzed to determine the deposition efficiency of silicone on the fabric. Digestion of the polydimethylsiloxane slurry indicates 2.46 wt % silicon element in the slurry. Deposition results indicate an average of 247 micrograms per gram fabric silicon on fabric, or 89% deposition of the added silicone. A control sample (identical to the polydimethylsiloxane slurry described above, but no polyvinyl alcohol gel added) shows 52 microgram per gram silicon on fabric.

### Example 13

10.0 g of Luviskol K-90 polymer (BASF, Germany) is added to 40 g of polyethylene glycol 400 (Dow Chemical, Midland, Mich.) in a 4 oz. glass jar. The contents are heated at 80° C. overnight to yield a clear mixture. The work of adhesion of the sample is measured using a TA X2 Plus Texture Analyzer (Texture Technologies, Scarsdale, N.Y.) and is determined to yield a work of adhesion of 7.6 millijoules.

### Example 14

50 g of anionically modified polyvinyl alcohol (Nippon Gohsei of Japan) is added to 450 g of deionized water at 60° C., while agitating using an Ultra Turrax T-25 mixer at 19,000 RPM. The dispersion is allowed to deaerate for 2 hours at 60° C. To the solution of polyvinyl alcohol is added 1.25 g of a 10 wt % gluteraldehyde solution, followed by the addition of 12 g of 2.0M HCl while agitating. This solution is allowed to sit overnight. In a separate beaker, 7 grams of Urea, 0.5 g of resorcinol (Aldrich of Wisconsin, USA), and 0.4 g of ammonium chloride is dissolved in 90 g deionized water. Next, 20 g of Ludox HS-30 silica (Grace Davison of Maryland, USA) is added. Then, 80 g of beta-ionone (Firmenich of New Jersey, USA) is added to the homogeneous solution while agitating (3-blade, pitched turbine agitator IKA Laboretechnik at 300 RPM), to emulsify the beta-ionone. Adjust the pH of this slurry to 3.0 using 2.0M HCl. Place beaker in a constant temperature bath maintained at 70 degrees Centigrade. Add 18 g of 37 wt % formaldehyde solution to the beta ionone emulsion while agitating at 300 RPM. After 1 hour of reaction at 70° C., 94 g of the 9.7 wt % solution of cross-linked polyvinyl alcohol is added to the reaction beaker. The contents are allowed to react for an additional 2 hours at 70° C. The finished slurry is then spray dried using a co-current Niro spray dryer (3 ft diameter, 200° C. inlet temperature, 95° C. outlet temperature, 72 kg/hr air, centrifugal wheel atomizer with diameter of 2 inches spinning at 30000 RPM) to recover 38-75 micrometer sized particles. The resulting benefit agent containing delivery particles are tested for deposition on cotton terry fabrics according to the deposition test method described herein. The resulting deposition efficiency is 47%.

### Example 15

The following reaction is carried out without solvent. To 3.32 g of the polyethyleneimine, Lupasol WF, 1.96 g of the epoxide Denacol EX 141 is added. The mixture is stirred with an Ultraturrax mixer for 1 minute and then the mixture's temperature is maintained at approximately 60° C. for approximately 12 hours. A high viscous polyaminoalcohol material is obtained. The resulting material is suitable for use as a coating material for the benefit agent containing delivery particle of the present invention. Lupasol WF is obtained from BASF of Ludwigshafen, Germany, and Denacol EX 141 is obtained from Nagase ChemteX Corporation 1-1-17, Shinmachi, Nishi-ku, Osaka, Japan 550-8668.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A benefit agent containing delivery particle comprising:
a.) a core material comprising a benefit agent selected from the group consisting of perfume, silicone oils, waxes, hydrocarbons, higher fatty acids, essential oils, lipids, skin coolants, vitamins, sunscreens, glycerine, catalysts, bleach particles, silicon dioxide particles, malodor reducing agents, antiperspirant actives, cationic polymers and mixtures thereof; and
b.) one or more coatings that coat at least a portion of said core material, said one or more coatings having a total work of adhesion of at least 0.2 millijoules, a total percent of dissolution of less than about 80%, said benefit agent containing delivery particle having a benefit agent loss of less than about 70%.

2. The benefit agent containing delivery particle according to claim 1 said benefit agent containing delivery particle's coating having a storage modulus from about 5 Pascals to about 500 Pascals.

3. The benefit agent containing delivery particle according to claim 1 said benefit agent containing delivery particle comprising more than one coating.

4. The benefit agent containing delivery particle according to claim 1 wherein said one or more coatings comprises a material selected from the group consisting of polyvinyl alcohol, polyvinyl pyrrolidone, polyethylene glycol, polyamines, polysaccharides and modified polysaccharides, gel forming proteins, modified celluloses, carboxylic acid containing acrylic polymers, polyureas, polyurethanes, gelatin, gum arabic, urea crosslinked with formaldehyde, urea crosslinked with gluteraldehyde, chitin and chitosan and modified chitin and modified chitosan, sodium alginate, latexes, silicon dioxide, sodium silicates and mixtures thereof.

5. The benefit agent containing delivery particle according to claim 1 said benefit agent containing delivery particle comprising at least 1 weight % benefit agent.

6. The benefit agent containing delivery particle according to claim 1 said core material comprising a perfume, said benefit agent containing delivery particle comprising, based on total benefit agent containing particle weight, from about 20 weight % to about 70 weight % perfume.

7. The benefit agent containing delivery particle of claim 1 said benefit agent containing delivery particle comprising a material selected from dyes, pigments and mixtures thereof.

8. The benefit agent containing delivery particle of claim 1 said benefit agent containing delivery particle having a particle size of from about 12 microns to about 2,000 microns.

9. The benefit agent containing delivery particle of claim 1 said benefit agent containing delivery particle having a deposition efficiency of at least 20%.

10. The benefit agent containing delivery particle of claim 1 wherein said one or more coatings have a total work of adhesion of from about 0.3 millijoules to about 20 millijoules and a total percent of dissolution of less than about 20%; said benefit agent containing delivery particle having a benefit agent loss of less than about 20% and a deposition efficiency of at least 50%.

11. A cleaning composition comprising the benefit agent containing delivery particle of claim 1.

12. The cleaning composition of claim 10 said cleaning composition comprising, based on total cleaning composition weight, from about 0.1 weight % to about 3.0 weight % benefit agent containing delivery particle, said benefit agent containing delivery particle comprising, based on total benefit agent containing particle weight, from about 20 weight % to about 70 weight % of a perfume.

13. A cleaning composition comprising the benefit agent containing delivery particle of claim 8.

14. A fabric care composition comprising the benefit agent containing delivery particle of claim 1.

15. The fabric care composition of claim 14 said composition comprising, based on total cleaning composition weight, from about 0.1 weight % to about 3.0 weight % benefit agent containing delivery particle.

16. A fabric care composition comprising the benefit agent containing delivery particle of claim 10.

17. A process of making the benefit agent containing delivery particle of claim 1, said process comprising the step of applying one or more coatings having, a total work of adhesion of at least 0.2 millijoules and a total percent of dissolution of less than about 80%, to a core material to form a benefit agent containing delivery particle, said one or more coatings being applied in a sufficient amount to provide said benefit agent containing delivery particle with a benefit agent loss of less than about 70%.

18. A method of applying a benefit agent, said method comprising:
a.) contacting at least a portion of a situs with a benefit agent containing delivery particle of claim 1 and/or a composition comprising the benefit agent containing delivery particle of claim 1; and
b.) then optionally washing and/or rinsing said situs or said portion of said situs.

19. The composition of claim 1 wherein said core material comprises a shell and/or adsorbent, at least a portion of said benefit agent being encapsulated by said shell when said shell is present and at least a portion of said benefit agent absorbed into said adsorbent when said adsorbent is present.

20. A substrate comprising a benefit agent delivery particle according to claim 1, said substrate and benefit agent containing delivery particle having an interfacial surface tension ratio greater than 1.
